(19) United States
(12) Patent Application Publication
(10) Pub. No.: US 2010/0028968 A1
(43) Pub. Date: Feb. 4, 2010

(54) BIOMECHANICAL TREATMENT FOR OBESITY AND DIABETES

(76) Inventors: Clinton Rubin, Port Jefferson, NY (US); Stefan Judex, Port Jefferson, NY (US); Jeff Pessin, Port Jefferson, NY (US)

Correspondence Address: FISH & RICHARDSON PC, P.O. BOX 1022, MINNEAPOLIS, MN, 55440-1022, US

(21) Appl. No.: 12/300,958

(22) PCT Filed: May 17, 2007
(86) PCT No.: PCT/US07/69154

§ 371 Date: Sep. 3, 2009

Related U.S. Application Data
(60) Provisional application No. 60/801,325, filed on May 17, 2006.

(51) Int. Cl. C12N 13/00

(52) U.S. Cl. 435/173.1

(57) ABSTRACT

Methods of maintaining or improving the metabolic state of a subject, e.g., a human, are disclosed. The methods can include providing to the subject a low magnitude, high frequency mechanical signal on a periodic basis and for a time sufficient to maintain or improve the subject's metabolic state. The subject can be diagnosed as having or can be at risk of developing, an obesity-related medical condition, e.g., type 2 diabetes, cardiovascular disease, hypertension, rheumatoid arthritis, and breast cancer. The methods can include a step of identifying a suitable subject by evaluating a physiological parameter that reflects the metabolic state of the subject, e.g., visceral fat content, subcutaneous fat content, body mass index, and blood pressure.

### CROSS-REFERENCE TO RELATED APPLICATIONS

This application claims the benefit of the filing date of U.S. Provisional Application No. 60/801,325, which was filed on May 17, 2006. For the purpose of any U.S. application that may claim the benefit of U.S. Provisional Application No. 60/801,325, the contents of that earlier filed application are hereby incorporated by reference in their entirety.

### STATEMENT REGARDING FEDERALLY SPONSORED RESEARCH OR DEVELOPMENT

The U.S. Government may have certain rights in this invention pursuant to Grant No. AR 43498 awarded by the National Institutes of Health and Grant No. NAG 9-1499 awarded by the National Aeronautics and Space Administration.

### TECHNICAL FIELD

This disclosure describes a treatment for weight control or weight gain and for related conditions, such as diabetes, that is non-invasive and non-pharmaceutical. More particularly, we describe an intervention in which low level, high frequency mechanical signals are applied to subjects for the suppression of weight gain and for the treatment or prevention of other undesirable conditions. As a result of improved weight control and/or by independent means, the present treatments can maintain or improve insulin resistant states and inhibit conditions associated with obesity, such as cardiovascular disease and hypertension.

### BACKGROUND

Obesity and diabetes are prevalent in the United States and are becoming more prevalent in other countries. In the U.S. alone, these conditions affect millions of people and encumber billions in annual health care service costs. Despite significant public attention, effective pharmacologic interventions at any scale have proven elusive. Even control of obesity and diabetes has proven difficult, with perhaps the only common etiologic factor being a “sedentary lifestyle,” and the only common intervention being exercise. The need for new treatment and prevention strategies is apparent.

### SUMMARY

The information that follows is based, in part, on our discovery that applying brief periods of low-magnitude, high-frequency mechanical signals to a subject (e.g., on a daily basis) can suppress adipogenesis, improve the subject's metabolic state (e.g., by markedly reducing free fatty acids and/or triglycerides in liver, muscle and/or adipose tissue), and improve glucose tolerance. While the present methods are not limited to those that produce a particular cellular response, our data indicate that the benefits we have observed are not achieved by elevating the subject's metabolism, as might occur with exercise, but primarily by suppressing the differentiation of precursor cells into adipocytes, thus biasing progenitors against a commitment to fat and inhibiting the etiologic progression of certain diseases, including those directly pronounced by obesity.

Accordingly, the invention features methods of altering (e.g., reducing) a subject's weight or promoting the maintenance of a healthier weight; of reducing or suppressing the further accumulation of subcutaneous fat; of reducing or inhibiting the further incorporation of fat in muscle or internal organs; of reducing or suppressing the further accumulation of visceral fat around internal organs; and/or of inhibiting the development or progression of obesity and disorders correlated with either excess weight per se or an undesirable fat distribution (e.g., fat accumulation around internal organs). These outcomes can occur in the course of maintaining or improving a subject's metabolic state, which is discussed in more detail below. Regardless of whether the methods are described with respect to a particular physiological parameter (such as a subject's weight) or more generally as being applicable to metabolic state or to a suspected or diagnosed condition (e.g., diabetes), the methods can be carried out by providing to the subject a low-magnitude, high-frequency physical signal. The physical-based signal is preferably mechanical, but can also be another non-invasive modality (e.g., acceleration, electric fields, or transcutaneous ultrasound). The signal can be supplied on a periodic basis and for a time sufficient to achieve one or more of the outcomes described herein. For example, the signal can be supplied to reduce the amount of visceral or subcutaneous fat or to suppress the rate of its production. The signal can also be supplied to maintain or improve the subject's metabolic state as evidenced, for example, by the rate of carbohydrate metabolism or lipid metabolism. Because our data indicate these physical signals can influence the fate of mesenchymal stem cells, the present methods can also be used to help retain or restore bone marrow viability and to direct the controlled differentiation of stem cells, including those placed in cell culture, down specific pathways. Our data further indicate that the physical signals described herein can upregulate peroxisome proliferative activated receptors gamma (PPAR-γ) and downregulate arachidonate 15-lipoxygenase (Alox15), both of which are associated with lipid metabolism. The upregulation of PPAR-γ and/or the downregulation of Alox15 can therefore be used to assess the adequacy of a given physical signal, as can non-molecular level indicators such as weight, fat distribution, and BMI, and such evaluation methods are within the scope of the present invention. Where molecular level indicators, including those discussed here or others that indicate cellular differentiation, are assessed, one may do so in vitro or in cell culture. Expression levels may be assessed in samples (e.g., blood, fat, urine, or bone marrow samples) obtained from animals serving as animal models or from human patients.

The time of exposure to the physical signal can be brief, and the periodic basis on which it is applied may or may not be regular. For example, the signal can be applied almost exactly every so many hours (e.g., once every 4, 8, 12, or 24 hours) or almost exactly every so many days (e.g., at nearly the same time every other day, once a week, or once every 10-14 days). Our expectation is that a positive outcome (e.g., an improved body weight, fat distribution, metabolic indicator, or obesity-related disease risk) will correlate with the level of compliance. However, less than ideal compliance and/or irregular application of the signal (which can be self-applied) are expected to be at least somewhat effective as well. Thus, in various embodiments, signals can be applied to a subject daily, but at varied times of the day. Similarly, a subject may miss one or more regularly scheduled sessions and treatment may stop and resume again at a later point in time. The length of time the signal (e.g., a mechanical signal) is provided can also be highly consistent in each application (e.g., it can be consistently applied for about 2-60 minutes, inclusive (e.g., for about 1, 2, 5, 10, 12, 15, 20, 25 or 30 minutes) or it can vary from one session to the next. Any of the methods can further include a step of identifying a subject (e.g., a human) prior to providing the low-magnitude, high-frequency physical (e.g., mechanical) signal, and the identification process can include an assessment of weight, fat mass, fat distribution, body mass index, blood sugar, triglyceride or free fatty acid levels, and/or any of other indicators of a metabolic state. We may use the terms “subject,” “individual” and “patient” interchangeably. While the present methods are certainly intended for application to human patients, the invention is not so limited. Domesticated animals, including cats and dogs, can also be treated.

As noted above, the present methods encompass those for maintaining or improving the metabolic state of a subject (e.g., a human of any age; children, adolescents, and adults, including the elderly, can all be treated). The methods can, optionally, include a step by which one identifies a suitable subject and a step of providing to the subject a low magnitude, high frequency mechanical signal on a periodic basis and for a time sufficient to maintain or improve the subject's metabolic state. Where the optional identification step is included, one can evaluate a physiological parameter that reflects the metabolic state of the subject. The parameter can be, for example, the level, in the subject (e.g., a level in the subject's blood or urine) of: a triglyceride, a free fatty acid, a cholesterol, fibrinogen, C-reactive protein, hemoglobin A1c, insulin, glucose, a pro-inflammatory cytokine, or an adipokine. Other parameters, any of which can be assessed either alone or in combination, include visceral fat content, subcutaneous fat content, body mass index, weight, or blood pressure. As noted, the subject may be overweight or obese, or may have metabolic syndrome or an obesity-related condition. A determination as to these conditions may have been made by a physician or other health care professional (i.e., a subject may have been diagnosed as having one of these conditions or as being at risk therefor). As the present methods can be applied to maintain a condition (e.g., metabolic state, weight, or fat distribution), the subject may also be apparently healthy (e.g., with no sign of a metabolic disorder or weight disorder).

Where the subject has, or is at risk of developing, an obesity-related medical condition, the condition can be type 2 diabetes, cardiovascular disease (as evidenced, for example, by atherosclerosis), hypertension, arthritis (e.g., osteoarthritis or rheumatoid arthritis), cancer (e.g., breast cancer, a cancer of the esophagus or gastrointestinal tract (e.g., stomach cancer or colorectal cancer), endometrial cancer, or renal cell cancer), carpal tunnel syndrome, chronic venous insufficiency, daytime sleepiness, deep vein thrombosis, end stage renal disease, gallbladder disease, gout, liver disease, pancreatitis, sleep apnea, or urinary stress incontinence. The subject may also be a person who has had, or who is at risk of having, a cerebrovascular accident. Because these conditions are recognized as obesity-related medical conditions, a person who is overweight, and particularly grossly overweight or obese is, by virtue of that fact alone, at risk of developing one or more of these conditions.

Subjects amenable to treatment with the present methods may also have restricted mobility associated with, for example, joint pain, back pain, or paralysis. These circumstances may arise independently or may result from one or more obesity-related medical conditions. For example, joint pain or back pain may result from or may be exacerbated by arthritis.

The present methods can include assessing the levels of one or more of the parameters set out herein and comparing them on one or more occasions to recommended levels. An undesirable level can indicate that the subject would be amenable to treatment as described herein. In addition to the parameters described above, one can assess (e.g., to determine metabolic state) the subject's glucose tolerance, insulin resistance, visceral and/or subcutaneous fat content, weight, body mass index, and/or blood pressure. Such parameters can be assessed in the course of identifying a subject amenable to treatment and can be monitored at one or more times after treatment has begun. More specifically, a subject can be diagnosed as being overweight, being obese, having diabetes, being susceptible to adiposity, or having metabolic syndrome or a metabolic disease. The cause(s) of excess weight, when present, may be known or unknown. For example, patients suffering from weight gain and/or diabetes caused by restricted mobility (e.g., as a result of paralysis, arthritis, or a muscular or neurodegenerative disorder) or a drug (e.g., steroids, protease inhibitors, and/or antipsychotics used as a treatment of other maladies) can be treated with the methods described herein. As the invention is non-pharmacologically based, it is anticipated that it can also readily and safely be used to chronically suppress or delay the onset of childhood obesity, diabetes, or any other obesity-related medical condition. As noted, treating apparently healthy and/or non-overweight patients is within the scope of the present invention, and such treatment is applied to reduce the risk of weight gain, obesity, or a weight- or obesity-related condition.

Accordingly, the invention features methods of treating patients who are apparently healthy (e.g., patients who are not overweight, obese, diabetic or suffering from a metabolic syndrome or an obesity-related medical condition) to reduce the risk that they will develop a condition described herein, to delay its onset, or to impede its progression. Thus, “altering” a subject's metabolic state can be achieved by maintaining the subject's metabolic state or changing the expected progression as well as by improving one or more of the physiological parameters described herein. For example, patients who begin taking a steroid for treatment of other conditions often experience weight gain. The present methods can be applied to alter such a subject's metabolic state so that a given patient is less likely to gain weight or to gain less weight than expected. “Treating” a patient with the present methods encompasses improving their prognosis or expected outcome.

The physical signals can be characterized in terms of magnitude and/or frequency, and are preferably mechanical in nature, induced through the weightbearing skeleton or directly by acceleration in the absence of weightbearing. Useful mechanical signals can be delivered through accelerations of about 0.01-10.0 g, where “g” or “1 g” represents acceleration resulting from the Earth's gravitational field (1.0 g=9.8 m/s/s). Surprisingly, signals of extremely low magnitude, far below those that are most closely associated with strenuous exercise, are effective. These signals can be, for example, of a lesser magnitude than those experienced during walking. Accordingly, the methods described here can be carried out by applying 0.1-1.0 g (e.g., 0.2-0.5 g (e.g., about 0.2 g, 0.3 g, 0.4 g, 0.5 g or signals therebetween (e.g., 0.25 g))). The frequency of the mechanical signal can be about 5-1,000 Hz (e.g., 20-200 Hz (e.g., 30-90 Hz)). For example, the frequency of the mechanical signal can be about 5-100 Hz, inclusive (e.g., about 50-90 Hz (e.g., 50, 60, 70, 80, or 90 Hz) or 20-50 Hz (e.g., about 20, 30, or 40 Hz). A combination of frequencies (e.g., a “chirp” signal from 20-50 Hz), as well as a pulse-burst of physical information (e.g., a 0.5 s burst of 40 Hz, 0.3 g vibration given at least or about every 1 second) can also be used. The magnitudes and frequencies of the acceleration signals that are delivered can be constant throughout the application (e.g., constant during a 10-minute application to a subject) or they may vary, independently, within the parameters set out herein. For example, the methods can be carried out by administering a signal of about 0.2 g and 20 Hz at a first time and a signal of about 0.3 g and 30 Hz at a second time. Further, distinct signals can be used for distinct purposes or aims, such as reversing an undesirable condition and preventing or inhibiting its development. For example, one can treat a subject for 15 minutes per day with a 0.3 g, 45 Hz signal where the aim is to lose fat mass, and for 10 minutes per day with a 0.2 g, 45 Hz signal to prevent fat gain.

The physical signals can be delivered in a variety of ways, including by mechanical means by way of Whole Body Vibration through a ground-based vibrating platform or weight-bearing support of any type that contacts the subject directly (e.g., through bare feet) or indirectly (e.g., through padding, shoes, or clothing). The platform can essentially stand alone, and the subject can come in contact with it as they would with a bathroom scale (i.e., by simply stepping and standing on an upper surface). The subject can also be positioned on the platform in a variety of other ways. For example, the subject can sit, kneel, or lie on the platform. The platform may bear all of the patient's weight, and the signal can be directed in one or several directions. For example, a patient can stand on a platform vibrating vertically so that the signal is applied in parallel to the long axis of, for example, the patient's tibia, fibula, and femur. In other configurations, a patient can lie down on a platform vibrating vertically or horizontally. A platform that oscillates in several distinct directions could apply the signal multi-axially. Devices can also deliver the signal focally, using local vibration modalities (e.g., to the subject's abdomen, thighs, or back), as well as be incorporated into other devices, such as exercise devices. The physical signals can also be delivered by the use of acceleration, allowing a limb, for example, to oscillate back and forth without the need for direct load application, thus simplifying the constraints of local application modalities (e.g., reducing the build-up of fat in limb musculature following joint replacement).

Considering the role of exercise in suppressing obesity and diabetes, it is widely accepted that exercise is effective because it metabolizes calories that accumulate through the diet and regulates insulin production through physiologic control of sugar in the bloodstream. Thus, one could conclude that the regulatory influence of exercise on suppressing the onset of obesity and diabetes is achieved through increasing calorie expenditure and reducing hyperglycemia, respectively, and thus the more strenuous the exercise, the greater the physiologic benefit. Our work, however, leads us to conclude that short daily bouts of extremely low-level mechanical, high-frequency loading can suppress fat production and improve insulin tolerance by controlling cellular differentiation. Because results can be achieved in a short time, the accumulation of a physical signal does not appear to be required, and this is consistent with the triggering of a biologic response. This trigger may change under systemic distress, such as endocrinopathy, obesity, cancers, infectious and/or genetic diseases, and/or aging, but by ensuring the trigger threshold is passed by adjusting duration, it still will not require an accumulated signal to obtain the benefit of the invention.

Because such low level signals, well below the forces, impacts, and/or accelerations that are generated by activities such as walking, are effective, the equilibration of caloric intake by metabolic work does not appear to be required. This is counterintuitive, counter to conventional wisdom, and implies a unique (or, at least, previously unappreciated) biologic mechanism. When we considered our results in view of how other physiologic systems, such as sight, hearing and touch, perceive exogenous signals through a frequency-selective “window,” and readily saturate when the signals are too high (too bright, too loud or too heavy), it occurred to us that physical signals could influence systems in a manner that is not necessarily dependent on reacting to highly intense—and perhaps dangerous—physical signals, but instead that cell processes are particularly sensitive to exogenous signals within specific frequency bands, and that exposure to such signals can control cellular outcomes, including differentiation of adipocyte precursors such as mesenchymal stem cells. We believe the physical signals we have used suppress adiposity not by stimulating the adipose tissue per se, but by influencing adipocyte precursors to differentiate into cells other than fat cells. Our studies indicate that the conditions described herein, including excess body weight, including weight gain to the point of obesity, metabolic state, and obesity-related medical conditions can be treated by the biologic suppression of adipocytic differentiation pathways and that that suppression can be achieved through low-level physical signals.

In addition to the methods carried out on whole, intact, living subjects, the signals described herein can be used to influence the fate of a cell in cell culture. These methods can be carried out by administering to the cell a low magnitude, high frequency mechanical signal on a periodic basis and for a time sufficient to influence the fate of the cell such that it differentiates into a cell type different from the cell type it would be expected to differentiate into in the absence of the signal (e.g., in the absence of a low magnitude, high frequency mechanical signal). Differentiation into a fully mature cell type may occur, but is not a necessary outcome.

Any cell type, including human cells of various types, can be subjected to the present signals. The methods can be applied, for example, to stem cells or progenitor cells (e.g., embryonic stem or progenitor cells or adult stem or progenitor cells, including mesenchymal stem cells). The magnitude and frequency of the signal applied can be as described herein (e.g., the magnitude of a mechanical signal can be or can be about 0.01-10.0 g (e.g., about 0.2-0.5 g, inclusive) and the frequency of the mechanical signal can be about 5-1000 Hz (e.g., about 30-100 Hz). The duration of the signal application (i.e., the overall period of time the signal is applied) can be the same as that for intact subjects, but it may also vary from that (e.g., it may be shorter; the periodic basis can involve repetition of the signal every five to ten minutes, once or twice an hour, or on a daily or weekly basis).

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a graph showing the results of glucose tolerance tests in C3H.B6-6T obesity-prone mice and (control and treated with mechanical signal; mean±SD). The treated group was subjected to a signal of 0.2 g and 90 Hz for 15 minutes/day, 5 days per week. Glucose tolerance was analyzed at eight weeks into the protocol. There is a marked improvement in glucose tolerance after treatment.

FIG. 2 is a pair of images of a three-dimensional reconstruction of a region of the thoracic region of C3H.B6-6T obesity-prone mice (control and treated with mechanical signals). The treated group was exposed to mechanical signals at 0.2 g, 90 Hz for 15 minutes/day, 5 days per week, for 9 weeks. Fat content was determined two days before euthanasia. The amount of fat within the thoracic region is significantly lower in the treated mice.

FIG. 3 is a graph showing the results of a body mass analysis of BL6 control and mechanically treated mice fed a high-fat diet for 10 weeks. Ten-week-old male BL6 mice were treated for brief periods each day. There is a marked suppression of weight gain, despite the same food intake.

FIG. 4 is a pair of images of a coronal cross-sectional 3-D in vivo microCT scan of the abdominal region of a mechanically treated (VIB) and a control (CTRL) mouse after 11 weeks of whole body treatment (signal application) vs. control. As measured by microCT, VIB animals had 27.6% less body fat (subcutaneous and visceral) in the torso than CTRL (p<0.005). VIB had 22.5% less epididymal and 19.5% less subcutaneous fat than CTRL (p<0.01).

FIG. 5 is a graph depicting body mass (g) of control and vibrated mice (n=20 in each group) over the span of twelve weeks. No significant differences in average body mass were measured between the controls and vibrated animals. The vibrated animals were vibrated five days per week, fifteen minutes per day at a 90 Hz, 0.4 g peak-to-peak acceleration.

FIG. 6A is an image of a three-dimensional longitudinal reconstruction of subcutaneous and epididymal fat content through the midsection of the torso of a control mouse, performed in vivo at twelve weeks, using computed tomographic signal parameters specifically sensitive to fat.

FIG. 6B is an image of a three-dimensional longitudinal reconstruction of subcutaneous and epididymal fat content through the midsection of the torso of a vibrated mouse (vibrated five days per week, fifteen minutes per day at a 90 Hz, 0.4 g peak-to-peak acceleration), performed in vivo at twelve weeks, using computed tomographic signal parameters specifically sensitive to fat.

FIG. 6C is an image of a three-dimensional transverse reconstruction of subcutaneous and epididymal fat content through the midsection of the torso of a control mouse, performed in vivo at twelve weeks, using computed tomographic signal parameters specifically sensitive to fat.

FIG. 6D is an image of a three-dimensional transverse reconstruction of subcutaneous and epididymal fat content through the midsection of the torso of a vibrated mouse (vibrated five days per week, fifteen minutes per day at a 90 Hz, 0.4 g peak-to-peak acceleration), performed in vivo at twelve weeks, using computed tomographic signal parameters specifically sensitive to fat. The data presented in FIGS. 6A-6D shows that following twelve weeks of daily, 15 minute low-level mechanical signal, the average amount of fat within the torso is 26% lower than that of age-matched control animals.

FIG. 7A is a graph depicting fat volume as a function of body mass for the control mice (n=15). The control animals demonstrated a strong positive correlation between fat volume and weight (r²=0.70; p=0.0001).

FIG. 7B is a graph depicting fat volume as a function of body mass for vibrated mice (n=15; vibrated five days per week, fifteen minutes per day at a 90 Hz, 0.4 g peak-to-peak acceleration). The vibrated animals showed a weak correlation between fat volume and weight (r²=0.18; p=0.1). Considering identical food intake between groups represented in FIGS. 7A and 7B, the data in FIGS. 7A and 7B indicate that the mechanical signals suppressed adipogenesis.

FIG. 8A is a graph depicting the level of total triglycerides (mg) in adipose tissue in vibrated mice (dark grey) and control group (light grey). The vibrated animals were vibrated five days per week, fifteen minutes per day at a 90 Hz, 0.4 g peak-to-peak acceleration. Triglycerides were 21.2% lower in adipose tissue in the vibrated animals when compared with controls (p=0.3; n=8 in each group). Mean and standard deviations are shown.

FIG. 8B is a graph depicting the level of total triglycerides (mg) in liver in vibrated mice (dark grey) and control group (light grey). The vibrated animals were vibrated five days per week, fifteen minutes per day at a 90 Hz, 0.4 g peak-to-peak acceleration. Triglycerides were 39.1% lower in livers of the vibrated animals when compared with controls (p=0.022; n=12 in each group). Mean and standard deviations are shown.

FIG. 8C is a graph depicting the level of total non-esterified fatty acids (mmol) in adipose tissue in vibrated mice (dark grey) and control mice (light grey). The vibrated animals were vibrated five days per week, fifteen minutes per day at a 90 Hz, 0.4 g peak-to-peak acceleration. Non-esterified fatty acids were 37.2% lower in adipose tissue of the vibrated animals when compared with controls (p=0.014; n=8 in each group). Mean and standard deviations are shown.

FIG. 8D is a graph graph depicting the level of total non-esterified fatty acids (mmol) in livers of vibrated mice (dark grey) and control mice (light grey). The vibrated animals were vibrated five days per week, fifteen minutes per day at a 90 Hz, 0.4 g peak-to-peak acceleration. Non-esterified fatty acids were 42.6% lower in livers of the vibrated animals when compared with controls (p=0.023; n=12 in each group). Mean and standard deviations are shown.

### DETAILED DESCRIPTION

We further describe below the present methods for applying physical stimuli to subjects. These methods can be applied in, and are expected to benefit subjects in, a great variety of circumstances that arise in the context of, for example, maintaining or improving the subject's metabolic state. The methods can be carried out, for example, to affect overt manifestations of the metabolic state (e.g., to suppress weight gain, obesity and defined conditions such as diabetes), and they may also affect underlying physiological events (e.g., the suppression of free fatty acids and triglycerides in adipose, muscle and liver tissue or the maintenance of “healthy” levels of such agents).

The methods are based, inter alia, on our findings that even brief exposure to high frequency, low magnitude physical signals (e.g., mechanical signals), inducing loads below those that typically arise even during walking, have marked effects on suppressing adiposity, triglyceride and free fatty acid production, and provide a unique, non-pharmacologic intervention for the control of weight gain, obesity, diabetes, and other obesity-related medical conditions. The marked response to low and brief signals in the phenotype of a growing animal suggests the presence of an inherent physiologic process that has been previously unrecognized.

Metabolic State

Metabolism constitutes a series of chemical processes that occur inside living organisms, including single cells found in vivo or placed in cell culture, which are necessary to maintain energy and life. In regard to the higher order organisms, such as a humans, the metabolic state of a subject can be affected by, for example, the subject's having metabolic syndrome or a metabolic disease, being overweight or obese, being inactive, confined to bed, or having diabetes or another obesity-related medical condition. Conversely, a poor metabolic state can lead to restricted mobility or even paralysis.

A subject's metabolic state can be reflected by the level of one or more of the following components in the subject (e.g., in a sample obtained from the subject (e.g., from the bloodstream, urine, protoplasm and/or tissue): triglycerides, free fatty acids, cholesterol, fibrinogen, C-reactive protein, hemoglobin Alc, insulin, and various cytokines (e.g., adipokines such as leptin (Ob ligand), adiponectin, resistin, plasminogen activator inhibitor-1 (PAI-1), tumor necrosis factor-alpha (TNFα) and visfatin), including pro-inflammatory cytokines. Adipokines are believed to have a role in modifying appetite, insulin resistance and atherosclerosis, and they may be modifiable causes of morbidity in people with obesity. A subject's metabolic state can also be reflected by glucose tolerance, insulin resistance, fat content (e.g., visceral or total fat), weight, body mass index, and/or blood pressure.

The present methods require application of a signal to a subject, and they can also, optionally, include a step of identifying a suitable subject. This step is optional because our research indicates that virtually anyone can benefit from the present methods, which can help maintain (i.e., impede a worsening of) the subject's current metabolic state, and that is true of subjects who are in excellent health. Where a subject's metabolic state is “reflected by” a given physiological parameter (or parameters), that parameter (or those parameters) can be evaluated, quantitatively or qualitatively, and this assessment can be used as a further indication of which subjects may be most likely to immediately benefit from the present methods or benefit to a greater extent. For example, where a subject's quality of life is negatively impacted by excessive weight, and where the present methods reduce or help to reduce that weight, that subject would be more immediately benefited than (and more greatly benefited than), for example, a subject who is only slightly overweight or who has been able to maintain a healthy weight.

The methods described here can be used to maintain or improve the metabolic state and are carried out by providing, to the subject, a low-magnitude and high-frequency physical signal, such as a mechanical signal. As noted, the physical signal can be administered other than by a mechanical force (e.g., an ultrasound signal that generates the same displacement can be applied to the subject), and the signal, regardless of its source, can be supplied (or applied or administered) on a periodic basis and for a time sufficient to maintain, improve, or inhibit a worsening of the metabolic state generally or to maintain, improve, or inhibit a worsening of a specific condition described herein (e.g., insulin resistance, obesity, diabetes or other obesity-related medical condition, or adipogenesis).

Subjects with Metabolic Syndrome

Metabolic syndrome, which is also called obesity syndrome, syndrome X, or insulin resistance syndrome, presents as a combination of metabolic risk factors. These factors include: weight gain, hypertension, atherogenic dyslipedemia (blood fat disorders, such as high triglycerides, low and/or high density lipoproteins (LDL and/or HDL); high LDL cholesterol fosters plaque buildup in arteries), insulin resistance or glucose intolerance, pro-thrombotic state (e.g., high fibrinogen or plasminogen activator inhibitor-1 in the blood) and pro-inflammatory state (e.g., elevated C-reactive protein in the blood). Accordingly, any of these factors can be assessed as a relevant physiological parameter. Amounts of each of the substances listed above (e.g., LDLs) that are considered normal, or healthy, are known in the art. These amounts are usually specified within a range. Similarly, tests and methods for assessing the parameters listed above (e.g., glucose tolerance or intolerance and weight gain) are known in the art, and the results are recognizable by health care professionals as desirable (healthy) or undesirable (indicating a disease process (e.g., diabetes) or unhealthy metabolic state, including obesity.

Potential causes of metabolic syndrome include physical inactivity, aging, hormonal imbalance and genetic predisposition. Thus, these causes may also be considered when performing the present methods and considering or evaluating subjects for treatment. Left uncontrolled, metabolic syndrome can lead to increased risk of diabetes and heart disease. Where a patient is also obese, that patient is at risk of developing an obesity-related medical condition. Recommended management of the syndrome presently focuses on lifestyle changes such as weight loss, increased physical activity and healthy eating habits. Any of these can be practiced in connection with the present methods, as can any treatment for an obesity-related medical condition.

The methods described here can be used to maintain, improve, or prevent (e.g., by inhibiting onset) a condition described herein (e.g., to maintain a healthy weight or to improve a sign or symptom of an undesirable state, such as metabolic syndrome or an obesity-related medical condition) by providing to a subject a low-magnitude and high-frequency physical (e.g., mechanical) signal on a periodic basis. The signal is applied for a time sufficient to maintain, improve, or prevent the condition (e.g., to maintain a healthy weight or to improve a sign or symptom of metabolic syndrome or an obesity-related medical condition). As noted, the physical signal is believed to reduce or suppress adipogenesis, and it may do so by influencing cellular differentiation toward a non-adipocyte fate). As also noted, the methods can include a step of assessing one or more of the physiological parameters described above in order to identify a subject amenable to treatment (e.g., hormonal imbalance). The subject can present with evidence of metabolic syndrome or as apparently healthy (e.g., a subject can have normal insulin sensitivity and blood glucose but a family history of diabetes or a genetic predisposition to obesity, as described further below). Moreover, the methods described herein can serve to suppress the negative sequelae associated with dyslipedemia and obesity, including atherosclerosis, congestive heart failure, myocardial infarction, hypertension, sleep apnea, and arthritis.

Subjects Who Are Overweight or Obese

Generally, an individual is considered to be overweight if his or her weight is 10% higher than normal as defined by a standard height/weight chart. An individual is considered to be obese if his or her weight is 30% or more above what is considered normal by the height/weight chart or as calculated relative to an ideal Body Mass Index (BMI).

Obesity is characterized by an excessively high amount of body fat or adipose tissue. This condition is common and varies from individual to individual. Some differences among individuals are influenced by inherited genetic variations. Genetic factors have been implicated in maintenance of body weight and effectiveness of diet and exercise, and some of the genes that have been implicated in predisposition to obesity include: UCP2 (whose gene product regulates body temperature), LEP (whose gene product, leptin, acts on the hypothalamus to reduce appetite and increase the body's metabolism), LEPR (leptin receptor), PCSK1 (whose gene product, proprotein convertase subtilisin/kexin type 1, processes hormone precursors such as POMC), POMC (whose gene product, among other functions, stimulates adrenal glands), MC4R (whose gene product is a melanocortin 4 receptor) and Insig2 (whose gene product regulates fatty acid and cholesterol synthesis). Other genes, which have been associated or linked with human obesity phenotypes now number above 200. Obesity gene map databases are available on the worldwide web and genes and gene maps are described in the scientific literature (see, e.g., Perusse et al., Obesity Res. 13:381-490, 2005). Any of these factors can be taken into consideration when determining a subject's risk of obesity.

Obesity affects an individual's quality of life and carries an increased risk for several related syndromes that can reduce life expectancy. Obese children are more prone to develop Type 2 diabetes (Cara et al., Curr. Diab. Rep. 6:241-250, 2006), while overweight adults, not yet even obese, are more susceptible to chronic, debilitating diseases and increased risk of death (Adams, NEJM, NEJMoa055643, 2006). Due to dyslipedemia and hypercholesterolemia, obese individuals have a markedly increased risk of atherosclerosis, leading to coronary artery disease and myocardial infarction. In addition, a vast majority of obese individuals have associated hypertension that can lead to thickening of the left ventricular wall (left ventricular hypertrophy), a leading cause of congestive heart failure. It is also well-established that obesity is associated with a generalized inflammatory response, which in combination with the increased mass of an individual puts mechanical and immunological stress on the major joints in the body, leading to more severe and earlier onset of arthritis. Further, nearly all obese individuals display various degrees of sleep apnea, a condition in which normal breathing is interrupted during periods of sleep, resulting in oxygen depletion, restless sleep, and chronic fatigue. While exercise remains the most readily available and generally accepted means of curbing weight gain and the onset of type II diabetes, compliance is poor. As described elsewhere herein, by reducing obesity or the risk of obesity, the present methods also reduce obesity-related medical conditions or the risk thereof.

Although obesity results in states of dyslipidemia, lipodystrophy (the absence of adipose tissue deposits) can have the same negative consequence due to limited peripheral nonesterified free fatty acids (NEFA) and triglyceride storage capacity (Petersen and Shulman, Am. J. Med. 119:S10-S16, 2006). Thus, a physiologic balance between lipid storage and lipid release must be maintained for optimum metabolism. The ability to suppress adipose tissue expansion by mechanical signals described herein, as well as to limit NEFA and triglyceride production (see, e.g., Example 3 infra), may provide a simple, non-pharmacologic approach to limit obesity in a manner sufficient to prevent the consequences of dyslipidemia.

The methods described herein can be used to treat an overweight or obese subject by providing to the subject a low-magnitude, high-frequency physical signal, preferably mechanical in origin, on a periodic basis and for a time sufficient to maintain or improve the subject's condition (e.g., reduce or suppress adipogenesis). In identifying a subject amenable to treatment, the methods can include a step of analyzing one or more of the genes listed or referenced above, or of assessing a subject's weight or predisposition for obesity by other methods known in the art. Because the signal does not required drug administration to be effective, this treatment described herein can also be safely administered to a juvenile and young-adult population to suppress childhood obesity and/or juvenile diabetes.

Subjects with Diabetes or Other Obesity-Related Medical Conditions

Diabetes mellitus is a disease in which the body does not produce or properly use insulin, a hormone that converts sugar, starches and other foods into energy. People with diabetes have a high circulating blood sugar level. Both genetics and environmental factors, such as obesity and lack of exercise, can play a role in the development and pathogenesis of diabetes.

There are generally considered to be four major types of diabetes: Type 1, Type 2, gestational and pre-diabetes. Type 1 Diabetes is an autoimmune disorder and results from the body's failure to produce insulin. Type 2 Diabetes results from the body's developed resistance to insulin, combined with relative insulin deficiency. Gestational diabetes affects pregnant women. Pre-diabetes is a condition in which a person's blood glucose levels are higher than normal but not high enough for a diagnosis of Type 2 Diabetes.

About 18 regions of the genome have been linked with Type 1 Diabetes risk (see, e.g., Dean et al., “The Genetic Landscape of Diabetes”, which is published online by the National Center for Biotechnology Information (NCBI)). These regions, each of which may contain several genes, have been labeled IDDM1 to IDDM18. The most well-studied is IDDM1, which contains the HLA genes that encode immune response proteins. There are two other non-HLA genes which have been identified thus far. One, IDDM2, is the insulin gene, and the other maps close to CTLA4, which has a regulatory role in the immune response.

Development of Type 2 Diabetes is associated with both genetics and environmental factors (see Dean et al.). Some genes implicated in developing Type 2 Diabetes encode: the sulfonylurea receptor (ABCC8), the calpain 10 enzyme (CAPN10), the glucagon receptor (GCGR), the enzyme glucokinase (GCK), the glucose transporter (GLUT2), the transcription factor HNF4A, the insulin hormone (INS), the insulin receptor (INSR), the potassium channel KCNJ11, the enzyme lipoprotein lipase (LPL), the transcription factor PPARgamma, the regulatory subunit of phosphorylating enzyme (PIK3R1) and others. These genes can be evaluated when identifying a subject who may benefit from the present methods.

Low-level mechanical signals described herein (see, e.g., Example 3 infra), can result in lower adiposity and suppress the production of nonesterified free fatty acids (NEFA) and triglycerides, key biochemical factors related to Type 2 diabetes. Numerous studies have demonstrated that dyslipidemia can have major negative impact on metabolism, growth and development. In particular, intra-tissue lipid accumulation (liver steatosis) and intra-myocellular lipids have been closely linked to insulin resistance and is the best predictor for the future development of insulin resistance (Unger, Endocrinology 144:5159-65, 2003).

The methods of the invention can be used to maintain or improve symptoms of diabetes in a subject by providing to the subject a low-magnitude, high-frequency physical signal, preferably a mechanical signal, at least once and preferably on a periodic basis and for a time sufficient to maintain or improve diabetes (e.g., by reducing or suppressing adipogenesis). In identifying a subject amenable to treatment, the methods can include a step of analyzing one or more of the genes listed or referenced above, of assessing a subject's blood glucose, or by other methods known in the art for identifying a patient who is diabetic or pre-diabetic. Similarly to the prevention and treatment of obesity, because this treatment is not based on the use of drugs, it can safely be used as an intervention in pre-adolescents and adolescents in the prevention and treatment of juvenile diabetes.

A subject who has been diagnosed as having, or is at risk of developing, another obesity-related medical condition can be treated as described herein. Other obesity-related medical conditions include cardiovascular disease, hypertension, osteoarthritis, rheumatoid arthritis, breast cancer, a cancer of the esophagus or gastrointestinal tract, endometrial cancer, renal cell cancer, carpal tunnel syndrome, chronic venous insufficiency, daytime sleepiness, deep vein thrombosis, end stage renal disease, gallbladder disease, gout, liver disease, pancreatitis, sleep apnea, a cerebrovascular accident, and urinary stress incontinence.

Adipogenesis

Adipogenesis, also called lipogenesis, is the formation of fat, including transformation of nonfat food materials into body fat. Adipogenesis also refers to the development of fat cells from preadipocytes.

The methods of this invention can be used to suppress or reduce adipogenesis in a subject (e.g., a human) by providing to the subject a low-magnitude, high-frequency physical signal (e.g., a mechanical signal) on a periodic basis and for a time sufficient to reduce or suppress adipogenesis. Subjects amenable to this treatment can include those diagnosed with being insulin resistant, overweight or obese, and at risk of being overweight or obese. The subjects can also be those diagnosed as having diabetes or metabolic syndrome.

Low-Magnitude High-Frequency Mechanical Signals

The treatments disclosed herein are unique, non-pharmacological interventions for a number of diseases or conditions, including obesity and diabetes.

The physical stimuli delivered to a subject (e.g., a human), can be, for example, vibrations, magnetic fields, and ultrasound. The stimuli can be generated with appropriate means known in the art. For example, vibrations can be generated by transducer(s) (e.g., actuator(s), e.g., electromagnetic actuator(s)), magnetic field can be generated with Helmholtz coil(s), and ultrasound can be generated with piezoelectric transducer(s).

The physical stimuli, if introduced as mechanical signals (e.g., vibrations), can have a magnitude of at least or about 0.01-10.0 g. As demonstrated in the Examples below, signals of low magnitude are effective. Accordingly, the methods described here can be carried out by applying at least or about 0.1-1.0 g (e.g., 0.2-0.5 g, inclusive (e.g., 0.2 g, 0.3 g, or 0.4 g)) to the subject. The frequency of the mechanical signal can be at least or about 5-1,000 Hz (e.g., 15 or 20-200 Hz, inclusive (e.g., 30-90 Hz (e.g., 30, 35, 40, 45, 50, or 55 Hz)). For example, the frequency of the mechanical signal can be about 5-100 Hz, inclusive (e.g., about 40-90 Hz (e.g., 50, 60, 70, 80, or 90 Hz) or 20-50 Hz (e.g., about 20, 25, 30, 35 or 40 Hz), a combination of frequencies (e.g., a “chirp” signal from 20-50 Hz), as well as a pulse-burst of mechanical information (e.g., a 0.5 s burst of 40 Hz, 0.3 g vibration given at least or about every 1 second during the treatment period). The mechanical signals can be provided on a periodic basis (e.g., weekly or daily). The physical signals can last at least or about 2-60 minutes, inclusive (e.g., 2, 5, 10, 15, 20, 30, 45, or 60 minutes).

Providing low-magnitude, high-frequency mechanical signals can be done by placing the subject on a device with a vibrating platform. An example of a device that can be used is the JUVENT 1000 (by Juvent, Inc., Somerset, N.J.) (see also U.S. Pat. No. 5,273,028). The source of the mechanical signal (e.g., a platform with a transducer, e.g., an actuator, and source of an input signal, e.g., electrical signal) can be variously housed or situated (e.g., under or within a chair, bed, exercise equipment, mat (e.g., a mat used to exercise (e.g., a yoga mat)), hand-held or portable device, a standing frame or the like). The source of the mechanical signal (e.g., a platform with a transducer, e.g., an actuator and a source of an input signal, e.g., electrical signal) can also be within or beneath a floor or other area where people tend to stand (e.g., a floor in front of a sink, stove, window, cashier's desk, or art installation or on a platform for public transportation) or sit (e.g., a seat in a vehicle (e.g., a car, train, bus, or plane) or wheelchair). The signal can also be introduced through oscillatory acceleration in the absence of weightbearing (e.g., oscillation of a limb), using the same frequencies and accelerations as described above.

Electromagnetic field signals can be generated via Helmholtz coils, in the same frequency range as described above, and with in the intensity range of 0.1 to 1000 micro-Volts per centimeter squared. Ultrasound signals can be generated via piezoelectric transducers, with a carrier wave in the frequency range described herein, and within the intensity range of 0.5 to 500 milli-Watts per centimeter squared. Ultrasound can also be used to generate vibrations described herein.

The transmissibility (or translation) of signals through the body is high, therefore, signals originating at the source, e.g., a platform with a transducer and a source of, e.g., electrical, signal, can reach various parts of the body. For example, if the subject stands on the source of the physical signal, e.g., the platform described herein, the signal can be transmitted through the subject's feet and into upper parts of the body, e.g., abdomen, shoulders etc.

As described in the Examples below, high frequency, low magnitude mechanical signals were delivered to mice via whole body vibration. When considering the potential to translate this to the clinic, it is important to note that associations persist between vibration and adverse health conditions, including low-back pain, circulatory disorders and neurovestibular dysfunction (Magnusson et al., Spine 21:710-17, 1996), leading to International Safety Organization advisories to limit human exposure to these mechanical signals (International Standards Organization. Evaluation of Human Exposure to Whole-Body Vibration. ISO 2631/1. 1985. Geneva). At the frequency (90 Hz) and amplitude used in the studies described herein (0.4 g peak-to-peak), the exposure would be considered safe for over four hours each day.

A number of embodiments have been described. Nevertheless, it will be understood that various modifications may be made without departing from the spirit and scope of the disclosure.

### EXAMPLES

### Example 1

### Biomechanical Treatment Improves Glucose Tolerance and Reduces Fat Content in Mice Prone to Obesity

C3H.B6-6T mice, bred as a congenic strain, have reduced (about 20%) circulating IGF-1 (insulin-like growth factor-1) and are phenotypically prone to obesity, despite being smaller than B6 mice. The congenic mice have reduced (by approximately 20%) circulating IGF-I (C3H.B6-6T [6T]) and were generated by backcrossing a small genomic region (30 cM) of chromosome 6 (Chr6) from C3H/HeJ (C3H) onto a C57B1/6J (B6) background. Thus, they are a unique strain, a “cross” of B6 and C3H.

Half of the C3H.B6-6T seven-week old female mice used in the study were treated by applying a mechanical signal at 0.2 g, 90 Hz for 15 min/day, while the other, untreated mice were used as controls. The five-days-per-week protocol was carried out for 9 weeks with the animals sacrificed at 16 weeks of age. Glucose tolerance was analyzed at eight weeks. Fat content of the thoracic cavity was determined two days before euthanasia by in vivo high-resolution micro-computed tomography scans (In Viva CT, Scanco, Inc.). Triglycerides (TG) and free fatty acid (FFA) were measured by extracting lipid from the serum, adipose tissue (peripheral/visceral), liver and the soleus muscle.

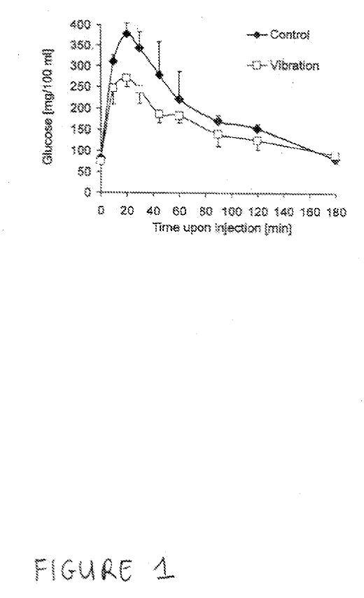

Glucose tolerance in the vibrated animals (analyzed at eight weeks) showed marked improvement in tolerance to insulin, as compared to controls (see FIG. 1).

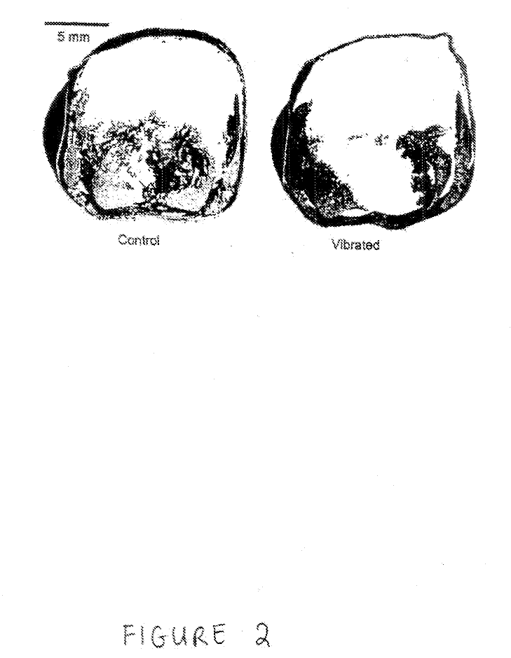

The in vivo scans of the thorax showed that the experimental animals had approximately 18% less volume of visceral fat than the controls (see FIG. 2).

Fasting glucose and insulin levels were unchanged between treated and control groups, suggesting that there was no significant effect on liver or beta cell function. The treated animals showed a 28% reduction in serum free fatty acids when compared to the controls. In the soleus muscle, the treated group showed 13% reduction in triglycerides and a 45% reduction in free fatty acids. In the adipose tissue, the vibrated group showed a 41% reduction in triglycerides and a 47% reduction in free fatty acids.

### Example 2

### Biomechanical Treatment Suppresses the Gain of Body Mass in Normal Mice Fed a High-Fat Diet and Normal Diet

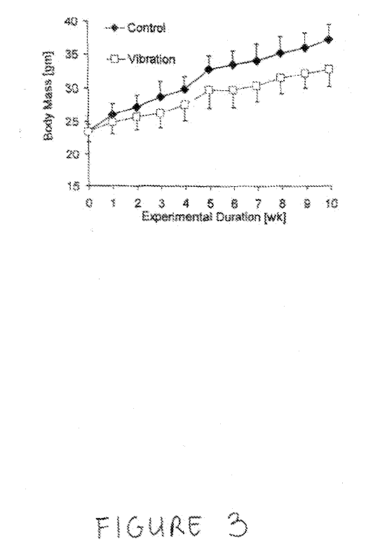

In a follow-up study using “normal” mice, 10-week-old C57BL/6J male mice (n=40) were fed a high-fat diet and treated by exposure to mechanical signals for a brief period each day. The treatment was carried out at 0.2 g, 90 Hz, as in Example 1. These mice showed a markedly lower body mass three weeks into the study than the controls (p<0.05 for all the remaining weeks), reaching a 13% difference at 10 weeks, despite identical food intake (see FIG. 3). At this point, total fat, summed for the entire torso, was 26% lower in the treated animals (p<0.007).

Vibrated mice fed a normal-fat diet were 8% lighter than controls at 10 weeks (p<0.05) and had 15% less body fat. Triglyceride and FFA levels were significantly reduced in the liver, adipose, and muscle tissues of these animals.

These data suggest that these biomechanical signals improve glucose tolerance and even reduce visceral fat content, indicating a unique, and perhaps interrelated, means of controlling long-term consequences of diabetes and obesity.

### Example 3

### Biomechanical Treatment Suppresses the Gain of Body Mass and Fat Content of Normal Mice Fed a Normal Diet

In one experiment, forty C57BL/6J male mice, 7 weeks old and fed a normal diet, were randomly separated into either a mechanically stimulated (MS) or control (CO) group. For 14 weeks, five days per week, the MS mice were subject to 15 minutes per day of a 90 Hz, 0.2 g whole body vibration induced via a vertically oscillating platform. A mechanical vibration at this magnitude and frequency is barely perceptible to human touch. Upon 12 weeks on their respective protocols (19 weeks of age), in vivo micro-CT scans were used to quantify subcutaneous and visceral fat of the torso (n=12 in each group). At sacrifice (21 weeks of age), weights of epididymal fat pad, subcutaneous fat pad, liver and heart were analyzed (all animals).

Following a 14 week exposure to short-duration, low-level whole body vibrations, food intake was 7.9% lower, and body mass was 6.7% lower as compared to control mice (p<0.05). In vivo CT measures indicated fat volume in the torso of the MS was 27.6% lower as compared to CO (p<0.005) (see FIG. 4). CT measures were directly supported by the weights of the dissected fat pads, where MS had 22.5% less epididymal and 19.5% less subcutaneous fat than CO (p<0.01). No difference in bone length or heart and liver weights was detected between the groups.

In yet another experiment, forty C57BL/6J male mice, seven weeks of age and fed ad libitum a normal rat chow diet, were randomly separated into one of two groups: those subjected to brief periods of whole body vibrations (WBV; n=20) or their age-matched sham controls (CTR; n=20). All procedures were reviewed and approved by the university's animal use committee. Animal weights, as well as their individual food consumption, were measured on a weekly basis. For fifteen weeks, five days per week, WBV mice were subject to fifteen minutes per day of a 90 Hz, 0.4 g peak-to-peak acceleration (1 g=earth's gravitational field, or 9.8m·s²), induced by vertical whole body vibration via a closed-loop feedback controlled, oscillating platform (modified DMT plate from Juvent, Inc, NJ) (Fritton et al., Ann. Biomed. Eng. 25:831-39, 1997). A sinusoidal vibration at this magnitude and frequency causes a displacement of approximately 12 microns and is barely perceptible to human touch. CTR animals were also placed on the vibrating platform each day, but the plate was not activated.

Twelve weeks into the protocol (animals at 19 w of age), in vivo micro-computed tomographic scans (VivaCT 40, Scanco Inc, SUI) were used to quantify fat and lean volume of the torso (n=15 in each group). The entire torso of each mouse was scanned at an isotropic voxel sixe of 76 microns (45 kV, 133 μA, 300 ms integration time), and noise was removed from the images with a Gaussian filter (sigma=1.5, support=3.0). The length of the torso was defined by two precise anatomical landmarks, one at the base of the pelvis and the other at the base of the neck. Image segmentation was calibrated using the density range of a freshly harvested fat pad from a B6 mouse unrelated to this study.

At 15 w into the protocol (22 w of age), eight mice from each group were fasted for 14-16 h prior to blood collection. Samples were collected by cardiac puncture with the animal under anaesthesia and the plasma separated by centrifugation (14,000 rpm, 15 min, 4° C.) and kept frozen until analysis. All mice were then killed by cervical dislocation and the different tissues (epididymal fat pad, subcutaneous fat pad, liver, and heart) quickly excised, weighed, frozen in liquid nitrogen and stored at −80° C. for further analyses.

Glycerol and insulin were measured in the plasma, and triglycerides (TG) and non-esterified free fatty acids (NEFA) were measured by extracting lipid from adipose tissue (n=8 per group) and liver (n=12 per group). Plasma insulin levels were measured using an ELISA kit (Mercodia Inc., Winston-Salem, N.C.). TG and NEFA from plasma and tissues were measured using enzymatic calorimetric kits: Serum Triglyceride Determination Kit (Sigma, Saint Louis, Mo.) and NEFA C (Wako Chemicals, Richmond, Va.), respectively. Total lipids from white adipose tissue (epididymal fat pad) and liver were extracted and purified following the chloroform-methanol method (Folch et al., J. Biol. Chem. 226:497-509, 1957) with some modifications, while liver glycogen content were determined by the anthrone method (Seifter et al., Arch. Biochem. 25:191-200, 950).

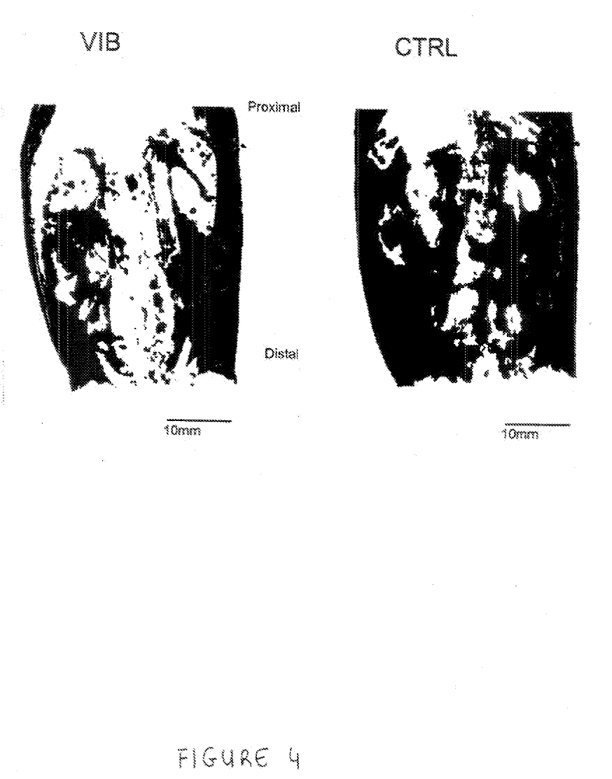

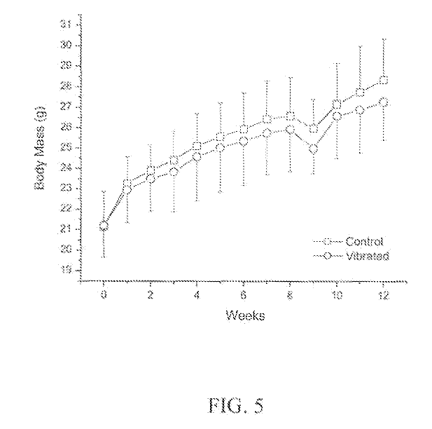

At baseline, body weights of WBV (21.1 g±1.7 g) and CTR (21.2 g±1.5) were similar (0.25% lower in WBV; p=0.9). Throughout the course of the protocol, weekly food intake between WBV (26.4 g·w⁻¹±2.1) and CTR (27.0 g·w⁻¹±2.1) was also similar (2.3% lower in WBV, p=0.3). Activity patterns during the fifteen minutes of sham (CTR) or vibration (WBV) treatment were not noticeably different from their behavior in their cages, or from each other. At 12 w, when the in vivo CT scans were performed, the body mass of WBV animals was not significantly different from CTR (4.0% lower in WBV, p=0.2; FIG. 5).

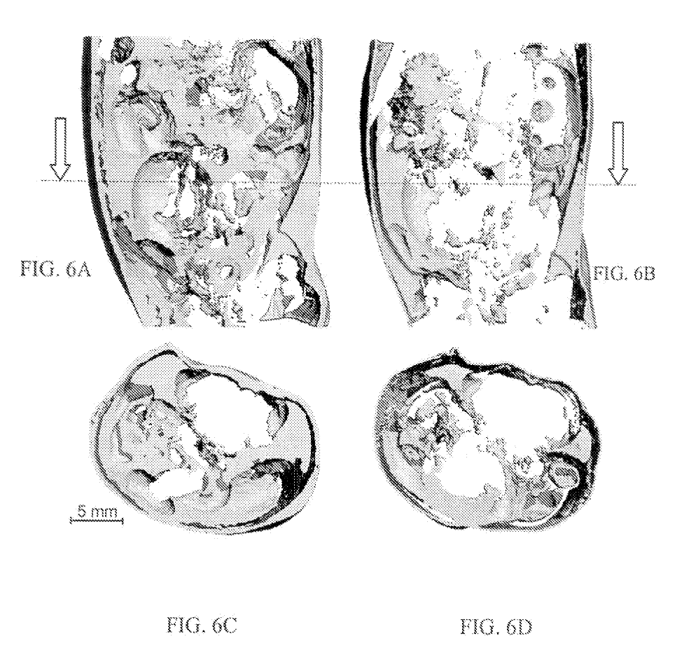

As measured at 12 w by in vivo CT, fat volume in the torso of WBV mice was 25.6% lower than that measured in CTR mice (p=0.01; FIGS. 6A-6D). In contrast, total lean volume of the torso was similar between WBV and CTR (p=0.7; Table 1 below), while lean volume as a ratio of body mass was 4.9% greater in WBV than CTR (p=0.01). Bone volume of the skeleton, from base of the skull to the distal region of the tibia, as a ratio of body mass was 5.9% greater in WBV than CTR (p=0.02). Fat volume normalized to body mass was 21.7% less in the WBV compared to controls (p=0.008). No differences in femoral length (p=0.6), the length of the torso (p=0.6), lean volume (p=0.5), heart (p=0.7) or liver weights (p=0.6), were measured between groups.

TABLE 1
Mean and standard deviation, as well as percentage difference and
p-values, of body habitus parameters at week 12 of the Control and
Vibrated mice, as defined by in vivo microcomputed tomography
(n = 15 in each group, p-values <0.05 are in bold).

| | | | % | |
|---|---|---|---|---|
| PARAMETERS | CONTROL | VIBRATED | DIFF | P |
| Body Mass @ 12 weeks (g) | 28.6 ± 2.49 | 27.4 ± 2.21 | −4.0 | 0.20 |
| Fat Volume (cm³) | 5.33 ± 1.67 | 3.96 ± 0.95 | −25.6 | 0.012 |
| Bone Volume (cm³) | 0.59 ± 0.07 | 0.60 ± 0.08 | +1.9 | 0.701 |
| Lean Volume (cm³) | 18.1 ± 1.3 | 18.3 ± 1.6 | +1.0 | 0.740 |
| Fat Volume/Body Mass (cm³/g) | 0.18 ± 0.04 | 0.14 ± 0.03 | −21.7 | 0.008 |
| Bone Volume/Body Mass (cm³/g) | 0.021 ± 0.001 | 0.022 ± 0.001 | +5.9 | 0.024 |
| Lean Volume/Body Mass (cm³/g) | 0.64 ± 0.03 | 0.67 ± 0.03 | +4.9 | 0.010 |
| Skeletal Length (cm) | 8.17 ± 0.20 | 8.21 ± 0.17 | +0.5 | 0.580 |
| Fat Volume/Skeletal Length (cm²) | 0.65 ± 0.19 | 0.48 ± 0.12 | −25.8 | 0.008 |
| Bone Volume/Skeletal Length (cm²) | 0.072 ± 0.008 | 0.073 ± 0.009 | +1.4 | 0.743 |
| Lean Volume/Skeletal Length (cm²) | 2.22 ± 0.13 | 2.23 ± 0.16 | +0.5 | 0.858 |
| Fat Mass (g) (density = 0.92) | 4.90 ± 1.54 | 3.64 ± 0.88 | −25.6 | 0.012 |
| Bone Mass (g) (density = 1.80) | 1.06 ± 0.13 | 1.08 ± 0.15 | +1.9 | 0.701 |

Fat volume data derived from in vivo CT were supported by the weights of the dissected fat pads performed post-sacrifice at 15 w, where WBV had 26.2% less epididymal (p=0.01) and 20.8% less subcutaneous (p=0.02) fat than CTR (Table 2 below). Normalized to mass, there was 22.5% less epididymal and 19.5% less subcutaneous fat in WBV than CTR (p=0.007).

TABLE 2
Mean and standard deviation, as well as percentage difference
and p-values, of body habitus (n ≧ 15 in each group)
and biochemical parameters (n = 8 in each group),
measured directly, post-sacrifice (n ≧ 15 in each
group, p-values <0.05 are in bold).

| | | | % | |
|---|---|---|---|---|
| PARAMETERS | CONTROL | VIBRATED | DIFF | P |
| Epididymal Fat weight | 0.63 ± 0.21 | 0.47 ± 0.12 | −26.2 | 0.014 |
| (g) | | | | |
| Subcutaneous Fat | 0.21 ± 0.06 | 0.17 ± 0.03 | −20.8 | 0.016 |
| weight (g) | | | | |
| Heart weight (g) | 0.120 ± 0.010 | 0.122 ± 0.015 | +1.6 | 0.707 |
| Liver weight (g) | 1.11 ± 0.11 | 1.09 ± 0.09 | −1.7 | 0.581 |
| Plasma Glycerol (mg/dL) | 17.37 ± 6.63 | 18.75 ± 9.31 | +7.9 | 0.64 |
| Plasma Insulin (ng/mL) | 0.54 ± 0.09 | 0.48 ± 0.07 | −10.8 | 0.068 |
| Plasma TG (mg/dL) | 38.74 ± 15.67 | 39.44 ± 12.4 | +1.8 | 0.89 |
| Plasma FFA (mmol/L) | 0.69 ± 0.32 | 0.63 ± 0.20 | −8.9 | 0.53 |

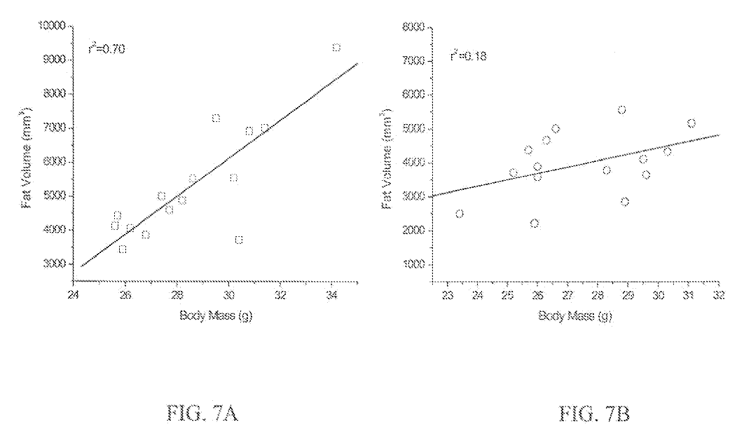

Correlations between food intake and either total body mass (r²=0.15; p=0.7) or fat volume (r²=0.008; p=0.6) were weak, and indicated that the lower adiposity in WBV animals could not be explained by differences in food consumption between the groups. While variations in body mass of the CTR mice correlated strongly with fat volume (r²=0.70; p=0.0001), no such correlation was observed in WBV (r²=0.18; p=0.1), indicating that fat mass contributed to weight gain in the controls, but failed to account for the increase in body mass in the mechanically stimulated animals (FIGS. 7A and 7B).

To account for the 1.2 g body mass difference between WBV and CTR mice measured at 12 w, in vivo CT measurements of fat volume were converted to mass equivalents. Using a density of 0.9196 g·cm⁻³ to convert fat volume to fat mass (Watts et al., Metabolism 51:1206-1210, 2002) indicated that the 3.64 g±0.9 of the average WBV mouse mass came from fat (13.3% of total mass), while 4.90 g±1.5 of the mass of the average CTR mouse came from fat (17.1% of total mass). Thus, the lack of fat in the WBV animals was, in essence, able to account for the “missing mass” between the groups (p=0.01).

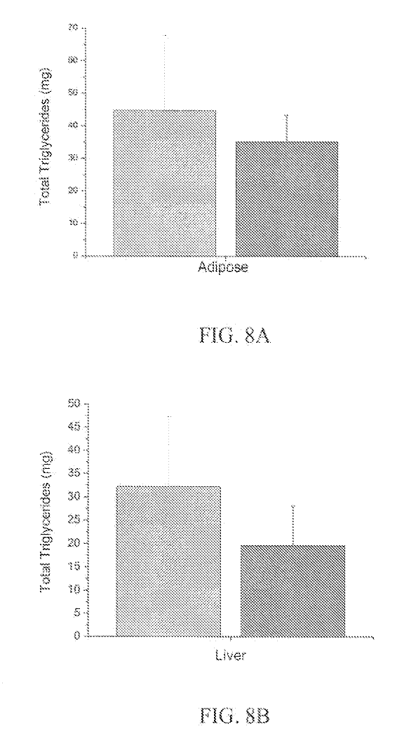

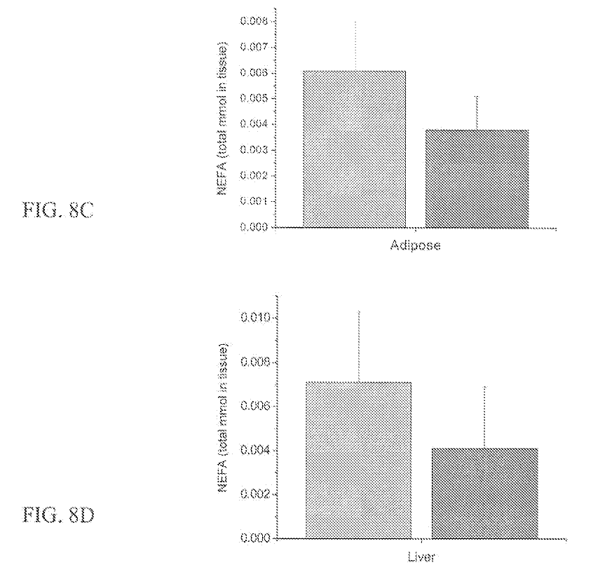

Fasting glucose and insulin levels showed only a trend in decreased plasma insulin in the WBV group (p=0.07), and taken together, these data suggested that these mechanical signals had no significant effect on liver or beta cell function (Table 2 above). At sacrifice, triglycerides (total mg in tissue) in adipose tissue of WBV were 21.1% (p=0.3) lower than CTR, and 39.1% lower in the liver (p=0.02; FIGS. 8A and 8B). Total non-esterified fatty acids (total mmol in tissue) in adipose tissue were 37.2% less in WBV as compared to CTR (p=0.01; FIG. 8C), while NEFA in the liver of WBV (total μmol/mg tissue) mice was 42.6% lower (p=0.02) than CTR (FIG. 8D). Glucose tolerance, tested at 9 w in three animals in each group, was slightly improved in WBV over CTR mice, but this difference was not statistically significant (data not shown).

In contrast to the perception that physical signals must be large and endured over a long period of time to offset caloric input and control insulin production, these results indicate that the cell population(s) and physiologic process(es) responsible for controlling fat mass and free fatty acid and triglyceride production are readily influenced by mechanical signals barely large enough to be perceived, an attribute achieved within an exceedingly short period of time.

The means by which these low-level signals suppress adiposity has been difficult to determine. Certainly, a trend towards improved glucose tolerance indicates that the metabolic machinery of the organism has been elevated, and remains higher long after the subtle challenge of low-level vibration has subsided, suggesting that a mechanosensory element within the cell population can be triggered without the signals necessarily being large (Rubin et al., Gene 367:1-16, 2006). And rather than requiring the accumulation of mechanical information through the product of time and intensity to elevate metabolic activity, perhaps these cell populations and physiologic processes are endowed with a memory, or refractory period, in which their metabolic machinery, once triggered, remains active even after the stimulus has subsided (Skerry et al., J. Orthop. Res. 6:547-551).

These data also suggest that mesenchymal cells are mechanically responsive, and that these physical signals need not be large to influence differentiation pathways. It appears that mesenchymal precursors perceive and respond to these mechanical “demands” as stimuli to differentiate down a musculoskeletal pathway, rather than “defaulting” to adipose tissue.

Other embodiments are within the scope of the following claims.

What is claimed is:

1-27. (canceled)

28. A method of influencing the fate of a cell in cell culture, the method comprising administering to the cell a low magnitude, high frequency mechanical signal on a periodic basis and for a time sufficient to influence the fate of the cell such that it differentiates into a cell type different from the cell type it would be expected to differentiate into in the absence of the low magnitude, high frequency mechanical signal.

29. The method of claim 28, wherein the cell is a stem cell or progenitor cell.

30. The method of claim 28, wherein the magnitude of the mechanical signal is about 0.01-10.0 g.

31. The method of claim 30, wherein the magnitude of the mechanical signal is about 0.2-0.5 g.

32. The method of claim 31, wherein the magnitude of the mechanical signal is about 0.3 g.

33. The method of claim 28, wherein the frequency of the mechanical signal is about 5-1000 Hz.

34. The method of claim 33, wherein the frequency of the mechanical signal is about 30-100 Hz.

35. The method of claim 34, wherein the frequency of the mechanical signal is about 90 Hz.

36. The method of claim 28, wherein the periodic basis is one every five to ten minutes, once or twice an hour, or on a daily or weekly basis.

37. The method of claim 28, wherein the time is about 10 seconds-200 minutes.
